(19) United States
(12) Patent Application Publication
(10) Pub. No.: US 2020/0405588 A1
(43) Pub. Date: Dec. 31, 2020

(54) TOPICAL TREATMENTS OF BATH TISSUES

(71) Applicant: GPCP IP Holdings LLC, Atlanta, GA (US)

(72) Inventor: Bruce J. Kokko, Neenah, WI (US)

(21) Appl. No.: 16/940,521

(22) Filed: Jul. 28, 2020

Related U.S. Application Data
(62) Division of application No. 14/330,082, filed on Jul. 14, 2014, now abandoned.
(60) Provisional application No. 61/866,739, filed on Aug. 16, 2013.

(51) Int. Cl. A61K 8/02; A61Q 19/00; A61K 8/37; A61K 8/34

(52) U.S. Cl. CPC A61K 8/0208; A61K 8/345; A61K 8/375; A61Q 19/00

(57) ABSTRACT

The present invention is directed to a tissue including a cellulosic substrate having a gel softener disposed on the substrate. The gel softener is a contact product of a gelator and a pharmaceutically or cosmetically acceptable oil. Further, the gel softener is sprayable at a temperature above room temperature and is in a solid or semi-solid state upon contact with the substrate at room temperature.

### CROSS REFERENCE TO RELATED APPLICATIONS

This application is Divisional of U.S. patent application Ser. No. 14/330,082, filed Jul. 14, 2014, which claims the benefit of U.S. Provisional Patent Application No. 61/866,739, filed Aug. 16, 2013, both applications are incorporated herein by reference in their entirety.

### TECHNICAL FIELD

The present invention is directed generally to bath tissue. More specifically, the present invention is related to topical treatments of bath tissue.

### BACKGROUND OF THE INVENTION

Softness is an important property of tissue products, such as bath tissues, facial tissues, and napkins. Accordingly, the commercial tissue industry uses a variety of approaches to increase tissue softness. For example, chemical debonders are incorporated into tissues to improve softness and tissue bulk. In addition, various production methods, such as creping and through air drying are used to enhance softness. Tissues treated with lotions during manufacture can be softer and provide skin soothing benefits.

However, the above mentioned industrial approaches have drawbacks. Chemical debonders can be expensive and are not conventionally biodegradable. Further, creping and through air drying can require high energy inputs. Although lotions can provide a skin moisturizing benefit, they can be too moist or greasy.

Based on the foregoing, there still exists a need for a tissue softener that is biodegradable, does not require high energy inputs, and imparts a moisturizing sensation without being overly wet or greasy. Accordingly, it is to solving this and other needs the present invention is directed.

### SUMMARY OF THE INVENTION

The present invention is directed to a tissue. In accordance with the present invention, the tissue includes a cellulosic substrate having a surface. The gel softener is disposed substantially on the surface of the substrate. The gel softener comprises the contact product of a gelator and a pharmaceutically or cosmetically acceptable oil. Yet, in another aspect, the gel softener is sprayable at a temperature above room temperature. Further, the gel softener is in a solid or semi-solid state upon contact with the substrate at room temperature.

In one aspect, the gelator is a sugar diester or 1,3:2,4 dibenzylidene sorbitol. In another aspect, the sugar diester has the following structure:

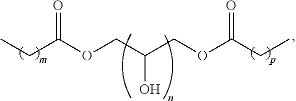

wherein n is an integer from 4 to 24, m is an integer from 6 to 32, p is an integer from 6 to 32, and m and p can be the same or different.

Yet, in another aspect, the sugar diester is a mannitol diester or a sorbitol diester. Still yet, in another aspect, the sugar diester is an octyl diester. The sugar diester can be a mannitol diester having the following structure:

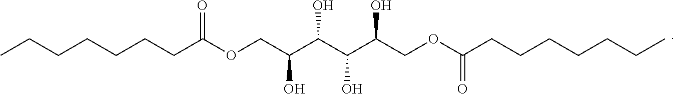 [SMILES: CCCCCCCC(=O)OC[C@H](O)[C@H](O)[C@@H](O)[C@@H](O)COC(=O)CCCCCCC]

In another aspect, the sugar diester is a sorbitol diester having the following structure:

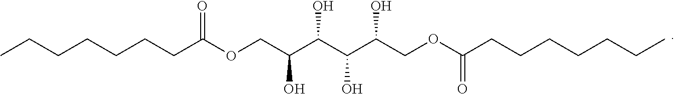 [SMILES: CCCCCCCC(=O)OC[C@@H](O)[C@H](O)[C@@H](O)[C@@H](O)COC(=O)CCCCCCC]

Yet, in another aspect, the gelator is 1,3:2,4 dibenzylidene sorbitol.

In one aspect, the gelator is present in the gel softener as a ratio of % weight to volume of oil (w/v) in a range between about 1.5% (w/v) and about 5.0% (w/v). The gel softener can be present on the cellulosic substrate at an add-on level in a range between about 1 pound per ton of tissue and about 60 pounds per ton of tissue. The oil can be an emollient, a humectant, a glycerol ester of fatty acids, a fatty acid alcohol, an essential oil, a C₁₂-C₁₅ alkyl benzoate ester, or any combination thereof.

In another aspect, the gelator can be a mannitol octyl diester, a sorbitol octyl diester or 1,3:2,4 dibenzylidene sorbitol. The mannitol octyl diester can have the following structure:

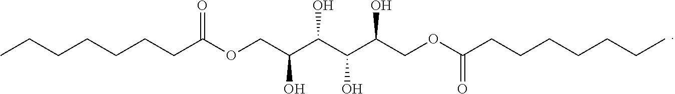 [SMILES: CCCCCCCC(=O)OC[C@H](O)[C@H](O)[C@@H](O)[C@@H](O)COC(=O)CCCCCCC]

The sorbitol octyl diester can have the following structure:

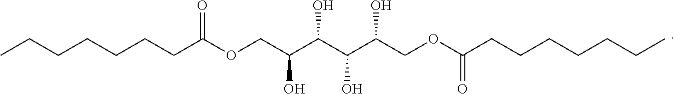 [SMILES: CCCCCCCC(=O)OC[C@@H](O)[C@H](O)[C@@H](O)[C@@H](O)COC(=O)CCCCCCC]

Yet, in another aspect, the gelator can be a sugar diester or 1,3:2,4 dibenzylidene sorbitol. In one aspect, the sugar diester has the following structure:

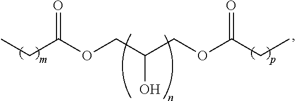

wherein n is an integer from 4 to 24, m is an integer from 6 to 32, p is an integer from 6 to 32, and m and p can be the same or different. The sugar diester can be a mannitol diester or a sorbitol diester.

A method of making a tissue includes forming a cellulosic substrate having a surface, mixing a gelator with a pharmaceutically or cosmetically acceptable oil at a temperature above room temperature to form a sprayable gel softener, and depositing the gel softener substantially on the surface of the cellulosic substrate. In one aspect, the gel softener gelates on contact with the cellulosic substrate which is at about room temperature. The gelator can be the mannitol octyl diester, the sorbitol octyl diester, or 1,3:2,4 dibenzylidene sorbitol, described above. In one aspect, the temperature of the sprayable gel softener is greater than about 105° C.

In another aspect, a method of making a tissue includes forming a cellulosic substrate and depositing a gel softener onto the substrate. The gel softener is sprayable at a temperature above room temperature and forms into a solid or semi-solid state at room temperature. The gel softener includes the contact product of a gelator and a pharmaceutically or cosmetically acceptable oil. The gelator can be a sugar diester or 1,3:2,4 dibenzylidene sorbitol. Further, the sugar diester can have the following structure:

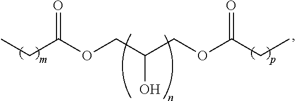

wherein n is an integer from 4 to 24, m is an integer from 6 to 32, p is an integer from 6 to 32, and m and p can be the same or different. In one aspect, the gel softener is formed on the substrate by spraying the oil onto the substrate to form an oil layer, and then spraying the sugar diester onto the oil layer to form the gel softener. In another aspect, the gel softener is formed on the substrate by mixing the 1,3:2,4 dibenzylidene sorbitol with the oil at a temperature above room temperature to form a solution, and then spraying the solution onto the substrate. Yet, in another aspect, the temperature of the gel softener is greater than about 105° C. The sugar diester can be a mannitol diester or a sorbitol diester.

It is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the present invention. It is important, therefore, that the claims be regarded as including such equivalent constructions insofar as they do not depart from the spirit and scope of the present invention.

Other advantages and capabilities of the invention will become apparent from the following description taken in conjunction with the examples showing aspects of the present invention.

### BRIEF DESCRIPTION OF THE DRAWINGS

The invention will be better understood and the above object as well as other objects other than those set forth above will become apparent when consideration is given to the following detailed description thereof. Such description makes reference to the annexed drawing wherein:

FIG. 1 illustrates the application of a gel softener onto a cellulosic substrate to form a tissue in accordance with the present invention.

### DETAILED DESCRIPTION OF THE INVENTION

For a fuller understanding of the nature and desired objects of this invention, reference should be made to the above and following detailed description taken in connection with the accompanying figures. When reference is made to the figures, like reference numerals designate corresponding parts throughout the several figures.

The term “cellulosic” as used herein refers to fibers or products incorporating papermaking fibers having cellulose as a major constituent. Suitable papermaking fibers include those derived from non-recycled paper sources, as well as secondary, recycled paper sources.

The term “consistency” as used herein refers to the percent solid in a composition comprising a solid in a liquid carrier. For example, the consistency of a fiber slurry weighing 100 grams and comprising 50 grams of fibers has a consistency of 50% by weight.

The term “sugar diester” as used herein refers to a compound having the following structure:

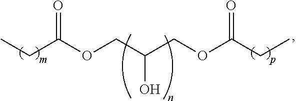

wherein n is an integer from 4 to 24, m is an integer from 6 to 32, p is an integer from 6 to 32, and m and p can be the same or different. Specific examples of the sugar diester include a mannitol diester and a sorbitol diester.

The term “gelator” as used herein refers to a sugar diester or 1,3:2,4 dibenzylidene sorbitol. The contact product of a gelator with a cosmetically or pharmaceutically acceptable oil forms a “gel softener.” The term “gel softener” is in a solid or semi-solid state at about room temperature. The term “room temperature” refers to a temperature from about 20° C. to about 25° C. The temperature of human skin is between about 30° C. and about 37° C.

The term “oil” as used herein refers to any cosmetically or pharmaceutically acceptable oil, for example any oil used in cosmetic or pharmaceutical compositions. Oils are hydrophobic chemical substances and can be natural, for example derived from vegetal and animal sources, synthetic, or semi-synthetic.

The terms “by weight” and “% by weight,” as used herein refer to weight of a given substance divided by the total weight of the tissue or gel softener, whichever is indicated. Typically, weight is measured in grams (g). For example, a composition with a total weight of 100 g, which includes 25 g of substance A, will include substance A in 25% by weight. The terms “% weight/volume” or “% (w/v)” as used herein refer to weight of a given substance divided by the volume of a second substance. Weight is measured in grams and volume is measured in milliliters (ml). For example, a composition including 0.1 g of substance A dissolved in 10 ml of water has 1% (w/v) of substance A.

The term “geometric tensile strength” or “GMT” refers to the strength of the tissue. GMT is calculated as the square root of the product of the machine direction (MD) tensile strength and the cross-machine direction (CD) tensile strength of the web. Geometric tensile strengths are measured using a MTS Synergy tensile tester or other suitable device using a 3 inch wide tissue sample, a jaw span of 2 inches, and a crosshead speed of 10 inches per minute after maintaining the sample under Technical Association of Paper and Pulp Industry (TAPPI) standard conditions for 4 hours before testing. A 50 Newton maximum load cell is utilized in the tensile test instrument.

Dry tensile strengths (MD and CD), stretch, ratios thereof, modulus, break modulus, stress and strain are measured with a standard Instron test device or other suitable elongation tensile tester. The test device or tensile tester may be configured in various ways, for example using 3 or 1 inch wide strips of tissue or towel, conditioned in an atmosphere of 23° C.±1° C. (73.4° F.±1° F.) at 50% relative humidity for 2 hours. The tensile test is run at a crosshead speed of 2 in/min. Tensile strength is sometimes referred to simply as “tensile” and is reported in breaking length (km) or g/3 inches (g/3 in.).

Sensory softness (SS) is determined by a panel of individuals trained to evaluate the softness of a tissue in terms of bulk and tactile softness. The individuals rank the tissue attributes according to a number scale, relative to controls. SS tests can be conducted, for example, as provided in European Patent Publication No. EP 1595027 A2, which is incorporated herein in its entirety by reference.

The present disclosure relates to a tissue product comprising a gel softener. The tissue can be any type of tissue product including a cellulosic substrate, such as a bath tissue, facial tissue, napkin, and the like. More specifically, a gel softener deposited on the cellulosic substrate improves the softness and smoothness of the tissue, compared to the tissue without the softener. At room temperature, the gel softener forms a solid or semi-sold layer on the tissue surface. Thus, the tissue provides a user with a cooling and refreshing effect when rubbed onto the skin. In contrast to a lotion-impregnated tissue, the tissue of the present disclosure does not leave the tissue with an overly moist or greasy feeling.

In accordance with the present invention, the tissue includes a cellulosic substrate having a surface. In one aspect, the gel softener resides or is disposed on the surface of the substrate with substantially no penetration into the web of the substrate. The gel softener includes the contact product of a gelator and a pharmaceutically or cosmetically acceptable oil. The gel softener is sprayable at a temperature above room temperature. Further, the gel softener is in a solid or semi-solid state upon contact with the substrate at room temperature.

The gel softener is thermally reversible and de-structures at a temperature greater than room temperature. In one aspect, the gel softener de-structures at a temperature greater than about 70° C. In another aspect, the gel softener de-structures at a temperature greater than about 80° C. Yet, in another aspect, the gel softener de-structures at a temperature greater than about 90° C. Still yet, in another aspect, the gel softener de-structures at a temperature greater than about 95° C. By proper choice of the oil, the gel softener can be tailored to de-structure at or near body temperature. When the gel softener de-structures at body temperature, the oil can transfer from the tissue to the user's skin upon rubbing the tissue on the skin.

In one aspect, the gelator is a sugar diester or 1,3:2,4 dibenzylidene sorbitol. In another aspect, the sugar diester is a mannitol diester or a sorbitol diester. The sugar diester can be a mannitol diester having the following structure:

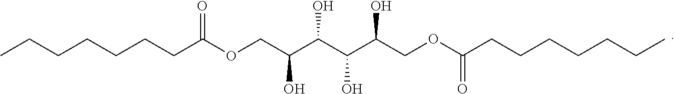 [SMILES: CCCCCCCC(=O)OC[C@H](O)[C@H](O)[C@@H](O)[C@@H](O)COC(=O)CCCCCCC]

In another aspect, the sugar diester is a sorbitol diester having the following structure:

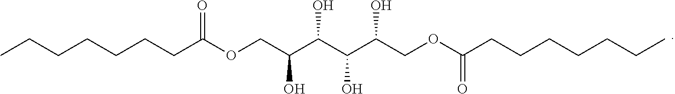 [SMILES: CCCCCCCC(=O)OC[C@@H](O)[C@H](O)[C@@H](O)[C@@H](O)COC(=O)CCCCCCC]

The above mannitol diester can be synthesized, for example, as described in Jadhav, S. R. et al., Sugar-Derived Phase-Selective Molecular Gelators as Model Solidifiers for Oil Spills, 49 Angew. Chem. Int. Ed. 1 (2010), which is incorporated herein in its entirety by reference. Yet, in another aspect, the gelator is 1,3:2,4 dibenzylidene sorbitol (sold as MILLAD 3905 by Milliken & Company, Spartanburg, S.C.).

In another aspect, a tissue made in accordance with the present invention includes a cellulosic substrate having a surface. The gel softener is disposed substantially on the surface of the substrate. Further, the gel softener includes the contact product of a gelator and a pharmaceutically or cosmetically acceptable oil. The gelator can be a mannitol octyl diester, a sorbitol octyl diester, or 1,3:2,4 dibenzylidene sorbitol. The mannitol octyl diester can have the following structure:

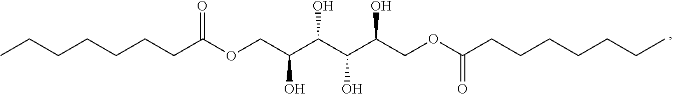 [SMILES: CCCCCCCC(=O)OC[C@H](O)[C@H](O)[C@@H](O)[C@@H](O)COC(=O)CCCCCCC]

and the sorbitol octyl diester can have the following structure:

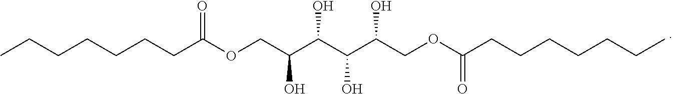 [SMILES: CCCCCCCC(=O)OC[C@@H](O)[C@H](O)[C@@H](O)[C@@H](O)COC(=O)CCCCCCC]

The gel softener is sprayable at a temperature above room temperature. Further, the gel softener is in a solid or semi-solid state upon contact with the substrate at room temperature.

Yet, in another aspect, a tissue includes a cellulosic substrate. A gel softener is disposed on the substrate. The softener is in a solid or semi-solid state at about room temperature and includes the contact product of a gelator and a pharmaceutically or cosmetically acceptable oil. The gelator can be a sugar diester or 1,3:2,4 dibenzylidene sorbitol. In one aspect, the sugar diester has the following structure:

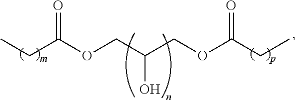

wherein n is an integer from 4 to 24, m is an integer from 6 to 32, p is an integer from 6 to 32, and m and p can be the same or different.

Still yet, in another aspect, a tissue includes a cellulosic substrate and a gel softener disposed on the substrate. The softener is in a solid or semi-solid state at room temperature. Further, the softener includes the contact product of a gelator and a pharmaceutically or cosmetically acceptable oil. The gelator can be a mannitol octyl diester, a sorbitol octyl diester or 1,3:2,4 dibenzylidene sorbitol. In one aspect, the mannitol octyl diester has the following structure:

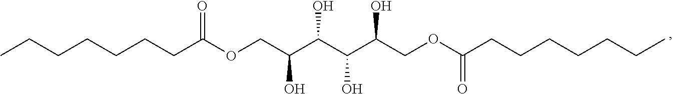 [SMILES: CCCCCCCC(=O)OC[C@H](O)[C@H](O)[C@@H](O)[C@@H](O)COC(=O)CCCCCCC]

and the sorbitol octyl diester having the following structure:

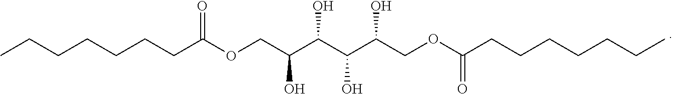 [SMILES: CCCCCCCC(=O)OC[C@@H](O)[C@H](O)[C@@H](O)[C@@H](O)COC(=O)CCCCCCC]

In another aspect, the gelator is the mannitol octyl diester.

The gelator is present in the gel softener as a ratio of % weight to volume of oil (w/v) in a range between about 1.5% (w/v) and about 5.0% (w/v). In one aspect, the gelator is present in the gel softener in a range between about 2.0% (w/v) and about 4.0% (w/v). Yet, in another aspect, the gelator is present in the gel softener in a range between about 3.0% (w/v) and about 5.0% (w/v).

The gel softener is present on the cellulosic substrate at an add-on level in a range between about 1 pound per ton of tissue and about 60 pounds per ton of tissue. In one aspect, the gel softener is present on the cellulosic substrate at an add-on level in a range between about 30 pounds per ton of tissue and about 40 pounds per ton of tissue. Yet, in another aspect, the gel softener is present on the cellulosic substrate at an add-on level in a range between about 40 pounds per ton of tissue and about 45 pounds per ton of tissue.

### Oil

The oil can be any pharmaceutically or cosmetically acceptable oil. The oil can be an emollient, a humectant, a glycerol ester of fatty acids, a fatty acid alcohol, an essential oil, a C₁₂-C₁₅ alkyl benzoate ester, or any combination thereof. Suitable polar emollients include, but are not limited to, alcohols, such as propylene glycol, glycerol, diethylene glycol, methylene glycol, polypropylene glycol, polyethylene glycol, sorbitol, and mixtures thereof. Suitable nonpolar emollients include, but are not limited to, aromatic or linear esters, such as Guerbet ester, C₁₂-C₁₅ alkyl benzoate esters, mineral oil, squalane, liquid paraffin, isopropyl myristate, tri-octyldodecyl-citrate, carnation oil, or mixtures thereof.

Suitable humectants include, but are not limited to, glycerol, hydrolyzed silk, ammonium lactate, hydroxypropyltrimonium hydrolyzed silk, hydroxypropyl chitosan, hydroxypropyltrimonium hydrolyzed wheat protein, lactamidopropyltrimonium chloride, ethyl esters of hydrolyzed silk, or combinations thereof.

Other examples of suitable oils include oils of vegetable origin, oils of animal origin, synthetic oils, semi-synthetic oils, or any combination thereof. Non-limiting examples of suitable oils include castor oil, olive oil, corn oil, carnation oil, soy bean oil, tubaki oil, cottonseed oil, sesame oil, avocado oil, jojoba oil, safflower oil, apricot oil, evening primrose oil, rose hip oil, grapeseed oil, coconut oil, carrot seed oil, eucalyptus oil, chamomile oil, neroli oil, tea tree oil, ylang ylang oil, spearmint oil, lavender oil, peppermint oil, sandalwood oil, squalane, mink oil, turtle oil, emu oil, cod liver oil, orange roughy oil, mink oil, polybutene, isopropyl myristate, isocetyl myristate, cetylisooctansate, isostearic acid, lauric acid, oleic acid, polyethylene glycol, polyethylene glycol 400, polyethylene glycol 860 monooleate, propylene glycol, glycerol, methylene glycol, polypropylene glycol, Guerbet ester, isostearyl alcohol, oleyl alcohol, cetyl alcohol, cetostearyl alcohol, stearyl alcohol, octamethylcyclotetrasiloxane, mineral oil, spindle oil, tamanu oil, or any combination thereof.

### Additives

Optionally, the gel softener includes additives. Non-limiting examples of suitable additives include wet strength agents, fragrances, emollients, surfactants, or any combination thereof.

The gel softener can include a fragrance. The fragrance can be present in an amount of from about 0.01% to about 2% by weight of the total weight of the gel softener. Suitable fragrances include volatile aromatic esters, non-aromatic esters, aromatic aldehydes, nonaromatic aldehydes, aromatic alcohols, non-aromatic alcohols, heterocyclic aroma chemicals, natural floral fragrances, such as blossom, carnation, gardenia, geranium, iris, hawthorne, hyacinth and jasmine, or any combinations thereof.

The gel softener can include a therapeutic amount of a medicinal agent. Medicinal agents include medicines, antipathogenic agents, antimicrobial agents, antibacterial agents, antiviral agents, disinfectants, analgesics, other types of medicine having suitable medicinal properties, and the like. For example, medicinal agents can be present in an amount of from about 0.01% to about 10% by weight of the total weight of the gel softener. Suitable antimicrobial agents include those effective against human pathogens, such as Escherichia coli, Staphylococcus aureus, Salmonella chloreraesuis, Salmonella typhi, Pseudomonas aeruginosa, Pseudomonas cepacia, and the Candida species, including Candida albicans. Specific suitable antimicrobial agents include 2,4,4′-trichloro-2′hydroxydiphenyl ether; 3,4,4′trichlorocarbanilide; 3,4,4′-trifiuoromethyl-4,4′-dichlorocarbanilide; 5-chloro-2-methyl-4-isothiazolin-3-one; iodopropynlbutylcarbamate; 8-hydroxyquinoline; 8-hydroxyquinoline citrate; 8-hydroxyquinoline sulfate; 4-chloro-3,5-xylenol; 2-bromo-2-nitropropane-1,3-diol; diazolidinyl urea; butoconazole; nystatin; terconazole; nitrofurantoin; phenazopyridine; acyclovir; clortrimazole; chloroxylenol; chlorhexidine; chlorhexidine gluconate; miconazole; terconazole; butylparaben; ethylparaben; methylparaben; methylchloroisothiazoline; methylisothiazoline; a mixture of 1,3-bis(hydroxymethyl)-5,5-dimethylhydantoin 50 and 3-iodo-2-propynyl butyl carbamate; oxyquinoline; EDTA; tetrasodium EDTA; p-hydroxyl benzoic acid ester; alkyl pyridinum compounds; quaternary ammonium compounds, such as coco phosphatidyl PG-dimonium chloride; or any combination thereof.

The gel softener can include a surfactant. Suitable surfactants include cationic surfactants, anionic surfactants, nonionic surfactants, amphoteric surfactants, or any combination thereof. The surfactant can be present in the gel softener in a range between about 10 wt. % to about 98 wt. % by weight of the total weight of the gel softener. Optionally, an anti-foaming compound can be included, for example octamethylcyclotetrasiloxane (sold as Anti-Foam by Dow Corning, Midland, Mich.).

Suitable amphoteric surfactants include, but are not limited to, betaines, sultaines, imidazoline derivatives, and the like. Non-limiting examples amphoteric surfactants include disodium cocoamphodiacetate, hydroxyethylglycinate cocoglucosides crosspolymer (sold as POLY SUGABETAINE C by Colonial Chemical, Inc., South Pittsburg, Tenn.), hydroxyethylglycinate laurylglucosides crosspolymer (sold as POLY SUGABETAIN L by Colonial Chemical, Inc.), cocamidopropyl betaine, stearyl betaine, stearyl amphocarboxy glycinate, sodium lauraminopropionate, cocoamidopropyl hydroxy sultaine, disodium lauryliminodipropionate, tallowiminodipropionate, cocoampho-carboxy glycinate, cocoimidazoline carboxylate, lauric imidazoline monocarboxylate, lauric imidazoline dicarboxylate, lauric myristic betaine, cocoamidosulfobetaine, alkylamidophospho betaine, or any combination thereof.

Examples of nonionic surfactants include, but are not limited to, glucosides, such as lauryl glucoside and decyl glucoside, and the ethoxylated alcohols and ethoxylates of long-chain, aliphatic, synthetic or native alcohols having a C₈-C₂₂ alkyl radical. These ethoxylated alcohols and can contain from about 1 to about 25 moles of ethylene oxide. The alkyl chain of the aliphatic alcohols can be linear or branched, primary or secondary, or saturated or unsaturated. Condensation products of C₁₀-C₁₈ alcohols with from about 2 to about 18 moles of ethylene oxide per mole of alcohol can be used. The alcohol ethoxylates can have a narrow homolog distribution (“narrow range ethoxylates”) or a broad homolog distribution of the ethylene oxide (“broad range ethoxylates”). Amide-fatty acid combinations, such as coconut amides, including cocamide diethanolamine and cocamide monoethanolamine, are also useful.

### The Cellulosic Substrate

The cellulosic substrate includes any fibers incorporating cellulose as a constituent. In one aspect, the cellulosic fibers are secondary, recycled fibers. In another aspect, the cellulosic fibers are derived from hardwood fibers, such as hardwood kraft fibers, hardwood sulfite fibers; softwood fibers, such as softwood kraft fibers, softwood sulfite fibers; or any combination thereof.

The cellulosic substrate can include a wet strength agent. The wet strength agent can be present in a range between about 0.05% and about 1.5% by weight of the total weight of the cellulosic substrate. The wet strength agent includes temporary, as well as permanent, wet strength agents. Non-limiting examples of suitable wet strength agents include glyoxal; glutaraldehyde; uncharged chemical moieties, such as dialdehydes, aldehyde-containing polyols, uncharged aldehyde-containing polymers, cyclic ureas, and aldehyde-containing cationic starch; mixtures of polyvinyl alcohol and salts of multivalent anions, such as boric acid or zirconium ammonium carbonates; glyoxalated polyacrylamide; polyamide-epichlorohydrin; polyamine-epichlorohydrin; ureaformaldehyde; melamine-formaldehyde; polyethyleneimine; or any combination thereof.

### Method of Making the Tissue

The cellulosic substrate can be prepared according to conventional processes known to those skilled in the art, including conventional wet pressing, through air drying, Yankee air drying, and variations thereof. The substrate can be creped or un-creped.

For example, conventional wet pressed tissues are prepared by first preparing and mixing the raw fiber material in a vat to produce a fiber slurry with a consistency of about 1% to about 5%. Then, the fiber slurry is diluted to a consistency of about 0.1% to about 1.0% and transferred through a centrifugal pump to a headbox. From the headbox, the fibrous mixture is deposited onto a moving foraminous wire, such as fourdrinier wire, to form a nascent web. Water can drain through the wire by use of vacuum and/or drainage elements. For drying on a Yankee dryer, first an adhesive material is sprayed onto the surface of the Yankee dryer. The nascent web is transferred onto the hot Yankee dryer via one or two press rolls. The web is dried on the Yankee dryer and then removed with a creping doctor, which scrapes the web from the surface of the Yankee dryer drum. Then, the dried web is wound into a roll at the reel of the paper machine.

The gel softener can be applied to the cellulosic substrate by any suitable method. The gel softener can be applied to one side or both sides of the cellulosic substrate. The gel softener can be applied to the cellulosic substrate during the paper conversion process. During paper conversion, a paper sheet from a jumbo reel is converted to a tissue paper, which includes a stage of embossing. In an additional step of conversion, several plies can be assembled together to form a multi-ply sheet. Following optional embossing or assembly, the converted sheet is guided to a station for winding and cutting to form individual rolls. Optionally, two or more plies are assembled. The gel softener can be applied to the cellulosic substrate after the Yankee dryer step, but before the conversion process. In one aspect, the gel softener is applied to the cellulosic substrate during or between any steps of the conversion process.

The gelator, the oil, or a combination thereof, can be deposited onto the cellulosic substrate by any method known in the art. Non-limiting examples of suitable methods include those disclosed in U.S. Patent Appl. Publication No. 2007/0124869, which is incorporated herein in its entirety by reference.

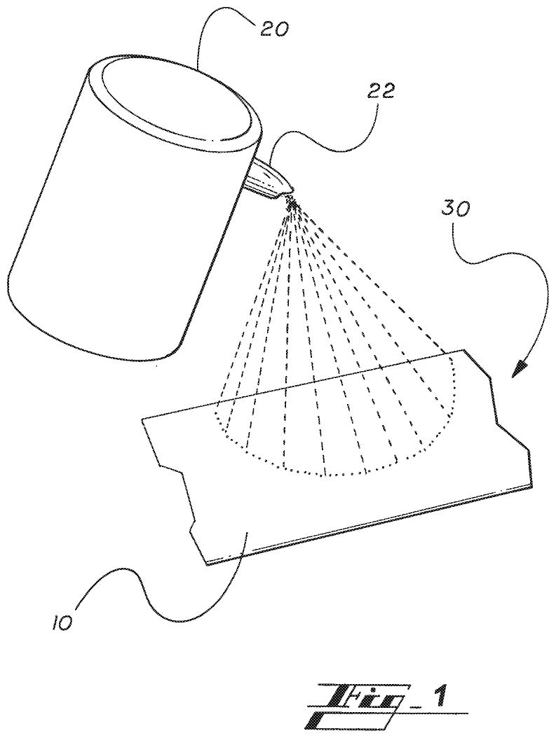

FIG. 1 illustrates the application of a gel softener onto a cellulosic substrate 10 to form a tissue 30 in accordance with the present invention. A tank 20 can house the gelator, the oil, or combination thereof, which can be deposited from a nozzle 22 onto the cellulosic substrate 10. Optionally, the tank 20 includes a temperature control to maintain the liquid solution at a selected temperature. In one aspect, the temperature control can maintain a temperature in a range between about 95° C. and about 110° C.

A method of making a tissue includes forming a cellulosic substrate having a surface, mixing a gelator with a pharmaceutically or cosmetically acceptable oil at a temperature above room temperature to form a sprayable gel softener, and depositing the gel softener substantially onto the surface of the cellulosic substrate. In one aspect, the gel softener gelates on contact with the cellulosic substrate and cooling to about room temperature. In another aspect, the temperature of the gel softener is greater than about 105° C.

In one aspect, a method of making a tissue includes forming a cellulosic substrate and depositing a gel softener onto the substrate. The gel softener is sprayable at a temperature above room temperature and forms into a solid or semi-solid state at room temperature. The softener includes the contact product of a gelator and a pharmaceutically or cosmetically acceptable oil. The gelator can be a mannitol octyl diester, a sorbitol octyl diester, or 1,3:2,4 dibenzylidene sorbitol. Further, the mannitol octyl diester can have the following structure:

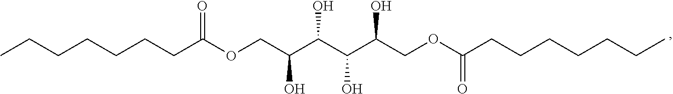 [SMILES: CCCCCCCC(=O)OC[C@H](O)[C@H](O)[C@@H](O)[C@@H](O)COC(=O)CCCCCCC]

and the sorbitol octyl diester can have the following structure:

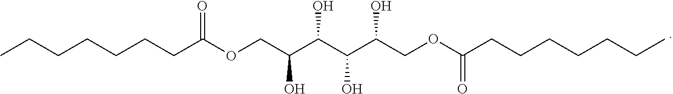 [SMILES: CCCCCCCC(=O)OC[C@@H](O)[C@H](O)[C@@H](O)[C@@H](O)COC(=O)CCCCCCC]

The gel softener can be formed on the substrate by spraying the oil onto the substrate to form an oil layer, and then spraying the mannitol octyl diester or the sorbitol octyl diester onto the oil layer to form the gel softener. In another aspect, the gel softener is formed on the substrate by mixing the 1,3:2,4 dibenzylidene sorbitol with the oil at a temperature above room temperature to form the gel softener, and then spraying the gel softener onto the substrate. Yet, in another aspect, the temperature of the gel softener is greater than about 105° C.

In another aspect, a method of making a tissue includes forming a cellulosic substrate and depositing a gel softener onto the substrate. The gel softener is sprayable at a temperature above room temperature and forms into a solid or semi-solid state at room temperature. The gel softener includes the contact product of a gelator and a pharmaceutically or cosmetically acceptable oil. The gelator can be a sugar diester or 1,3:2,4 dibenzylidene sorbitol. Further, the sugar diester can have the following structure:

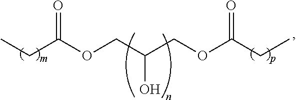

wherein n is an integer from 4 to 24, m is an integer from 6 to 32, p is an integer from 6 to 32, and m and p can be the same or different. In one aspect, the gel softener is formed on the substrate by spraying the oil onto the substrate to form an oil layer, and then spraying the sugar diester onto the oil layer to form the gel softener. In another aspect, the gel softener is formed on the substrate by mixing the 1,3:2,4 dibenzylidene sorbitol with the oil at a temperature greater than room temperature to form the gel softener, and then spraying the gel softener onto the substrate. Yet, in another aspect, the temperature of the gel softener is greater than about 105° C. The sugar diester can be a mannitol diester or a sorbitol diester. Further, the mannitol diester or the sorbitol diester can be an octyl diester. Yet, in another aspect, the sugar diester is a mannitol diester having the following structure:

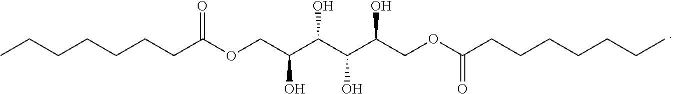 [SMILES: CCCCCCCC(=O)OC[C@H](O)[C@H](O)[C@@H](O)[C@@H](O)COC(=O)CCCCCCC]

Still yet, in another aspect, the sugar diester is a sorbitol diester having the following structure:

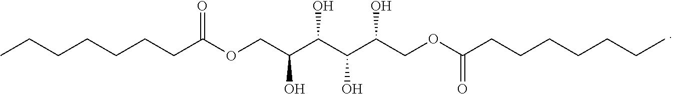 [SMILES: CCCCCCCC(=O)OC[C@@H](O)[C@H](O)[C@@H](O)[C@@H](O)COC(=O)CCCCCCC]

In one aspect, the gelator is 1,3:2,4 dibenzylidene sorbitol.

### EXAMPLES

### Example 1

A beaker was charged with 34.46 g polyethylene glycol 400 (PEG-400) (99 wt. %) and 0.35 g (1.00 wt. %) MILLAD 3905 and with vigorous stirring, heated to 125° C. to obtain a clear, thin solution. The solution became a viscous fluid upon standing and cooling to room temperature.

### Example 2

Example 1 was repeated with 0.70 g (2 wt. %) of MILLAD 3905. The clear, thin solution became a clear, viscous fluid upon standing and cooling to room temperature.

### Example 3

Example 1 was repeated with 1.05 g (2.96 wt. %) MILLAD 3905. The clear, thin solution became a clear, viscous fluid upon standing and cooling to room temperature, but with a little agitation, converted to a soft translucent gel (analogous to the consistency of Vaseline).

### Example 4

A beaker was charged with 39.29 g PEG-400 (97.49 wt. %) and 1.01 g (2.51 wt %) MILLAD 3905 and with vigorous stirring, heated to 125° C. to obtain a clear, thin solution. The solution became a product having a hazy top gel layer and a fluid bottom layer upon standing and cooling to room temperature.

### Example 5

A beaker was charged with 36.68 g polyethylene glycol monooleate (PEG-860-monooleate) (97.60 wt. %) and 0.90 g (2.40 wt %) MILLAD 3905, and with vigorous stirring, heated to 125° C. to obtain a clear, thin solution. The solution became a clear, translucent gel upon standing and cooling to room temperature.

### Examples 6-8

Example 5 was repeated for Examples 6-8 with 42.63 g PEG-860-monooleate and the amount of MILLAD 3905 indicated in Table 1. The resulting products after standing and cooling to room temperature are shown in Table 1.

TABLE 1
Composition of Examples 6-8

| | ^{†}MILLAD 3905 | ^{†}MILLAD 3905 | |
|---|---|---|---|
| Example | (g) | (wt %) | Product |
| 6 | 0.215 | 0.50 | fluid |
| 7 | 0.645 | 1.49 | fluid |
| 8 | 0.865 | 2.00 | gel |

^{†}MILLAD 3905: 1,3:2,4 dibenzylidene sorbitol

### Examples 9-12

A beaker was charged with 0.0152 g octamethylcyclotetrasiloxane (sold as Anti-Foam by Dow Corning, Midland, Mich.), 24.00 g water, 16.00 g sodium bis-hydroxyethylglycinate coco-glucosides crosspolymer (sold as POLY SUGABETAINE by Colonial Chemicals, South Pittsburg, Tenn.), the amount of propylene glycol (PG) indicated in Table 2, and 0.40 g MILLAD 3905. The mixture was heated to 105° C. while stirring vigorously, to yield a clear, thin solution. Upon standing and cooling to room temperature, the clear, thin solution yielded the product indicated in Table 2.

The gel obtained in Example 11 exhibited hysteresis with cycles of reheating (fluidizing) and re-cooling, and became opaque instead of translucent. In contrast, the gel in Example 12 showed little hysteresis, even after three successive heating/cooling cycles. Examples 9-11 all showed some hysteresis.

TABLE 2
Composition of Examples 9-12

| | MILLAD 3905 | | PG | |
|---|---|---|---|---|
| Example | (wt %) | ^{†}PG (g) | (wt %) | Product |
| 9 | 72 | 15.50 | 27.72 | Translucent gel |
| 10 | 74 | 13.99 | 25.71 | ^{‡}Translucent gel |
| 11 | 70 | 16.58 | 29.09 | Translucent gel |
| 12 | 66 | 20.00 | 33.10 | Translucent gel |

^{†}PG: propylene glycol
^{‡}Slightly hazy

### Example 13

The formulation of Example 12 was heated to 110° C. to obtain a clear solution. The solution was allowed to cool with continuous stirring. At 90° C., the fluid became thick and cloudy. At 35° C., partial gelation was observed. Under these conditions, only incomplete gelation was observed, even if allowed to stand at room temperature. However, the expected translucent gel was obtained when the product was reheated to form a clear solution, and then allowed to cool without agitation. Thus, cooling without agitation yielded the optimal gel.

### Example 14

A tissue was prepared in accordance with the present invention. A pressurizable container was charged with 1000.8 g of a 40% aqueous solution of POLY SUGABETAINE (26.48 wt. %), 0.36 g Dow Corning Anti-Foam (0.02 wt. %), 500.4 g (33.10 wt. %) propylene glycol, and 10.02 g (0.66 wt. %) MILLAD 3905. The solution was heated to 99° C. while stirring vigorously to yield a clear, thin solution. The resultant solution was kept at 95±2° C. while stirring to inhibit gelling. The solution was pressurized and coupled to a spray system fitted with 1650 SS air cap 62240-60°-SS nozzle (commercially available from Spring Systems, Inc., Valley Stream, N.Y.). Then the solution was sprayed onto Angel Soft® tissue (Georgia Pacific Company, Atlanta, Ga.) at a speed setting of 10, with air pressure of 20 psi, and a fluid pressure of 40 psi to achieve an add-on rate of 30-40 pounds per ton of tissue.

### Example 15

For comparison to Example 14, a tissue was prepared with a commercial blend of a silicone-based softener, WACKER TS50E (commercially available from Wacker Chemie, Munchin, Germany). The softener was sprayed onto Angel Soft® tissue as described above for Example 14 to achieve an actives add-on level of 45 pounds per ton of tissue.

The resultant tissues from Examples 14 and 15 were tested for geometric mean tensile strengths (GMT) and sensory softness (SS). As shown in Table 3, the tissues made in accordance with the present invention (Example 14) yielded significant increases in sensory softness compared to the tissues prepared with WACKER TS50E (Example 15), after correcting for tensile loss.

TABLE 3
Compositions of Examples 14 and 15

| | | Predicted Sensory | Actual | |
|---|---|---|---|---|
| Treatment | ΔGMT | softness (SS)^{†} | SS | ΔSS |
| Control | — | — | 17.8 | — |
| Example 14 | −133 | 18.4 | 18.7 | +0.3 |
| (MILLAD 3905) | | | | |
| Example 15 | −205 | 18.7 | 18.6 | −0.1 |
| (Wacker TS50E) | | | | |

^{†}Sensory softness changes were due solely to tensile strength changes

With respect to the above description, it is to be realized that the optimum proportional relationships for the parts of the invention, to include variations in components, concentration, shape, form, function, and manner of manufacture, and use, are deemed readily apparent and obvious to one skilled in the art, and all equivalent relationships to those illustrated in the specification are intended to be encompassed by the present invention.

Therefore, the foregoing is considered as illustrative only of the principles of the invention. Further, various modifications may be made of the invention without departing from the scope thereof, and it is desired, therefore, that only such limitations shall be placed thereon as are imposed by the prior art and which are set forth in the appended claims.

1. A method of making a tissue comprising:
forming a cellulosic substrate having a surface;
mixing a gelator with a pharmaceutically or cosmetically acceptable oil at a temperature above room temperature to form a sprayable gel softener, the gelator being a mannitol octyl diester, a sorbitol octyl diester, or 1,3:2,4 dibenzylidene sorbitol,
the mannitol octyl diester having the following structure: 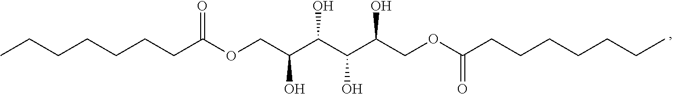 [SMILES: CCCCCCCC(=O)OC[C@H](O)[C@H](O)[C@@H](O)[C@@H](O)COC(=O)CCCCCCC]
and the sorbitol octyl diester having the following structure: 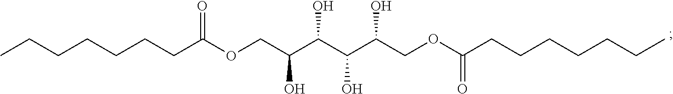 [SMILES: CCCCCCCC(=O)OC[C@@H](O)[C@H](O)[C@@H](O)[C@@H](O)COC(=O)CCCCCCC]
and depositing the gel softener substantially on the surface of the cellulosic substrate, the gel softener gelating on contact with the cellulosic substrate and cooling to about room temperature.

2. The method of claim 1, wherein the temperature of the gel softener is greater than about 105° C.

3. The method of claim 1, wherein the gelator is present in the gel softener as a ratio of % weight to volume of oil (w/v) in a range between about 1.5% (w/v) and about 5.0% (w/v).

4. The method of claim 1, wherein the gel softener is deposited onto the cellulosic substrate at an add-on level in a range between about 1 pound per ton of tissue and about 60 pounds per ton of tissue.

5. The method of claim 1, wherein the oil is an emollient, a humectant, a glycerol ester of fatty acids, a fatty acid alcohol, an essential oil, a C₁₂-C₁₅ alkyl benzoate ester, or any combination thereof.

6. The method of claim 1, wherein the oil is castor oil, olive oil, corn oil, carnation oil, soy bean oil, tubaki oil, cottonseed oil, sesame oil, avocado oil, jojoba oil, safflower oil, apricot oil, evening primrose oil, rose hip oil, grapeseed oil, coconut oil, carrot seed oil, eucalyptus oil, chamomile oil, neroli oil, tea tree oil, ylang ylang oil, spearmint oil, lavender oil, peppermint oil, sandalwood oil, squalane, mink oil, turtle oil, emu oil, cod liver oil, orange roughy oil, mink oil, polybutene, isopropyl myristate, isocetyl myristate, cetylisooctansate, isostearic acid, lauric acid, oleic acid, polyethylene glycol, polyethylene glycol 400, polyethylene glycol 860 monooleate, propylene glycol, glycerol, methylene glycol, polypropylene glycol, Guerbet ester, isostearyl alcohol, oleyl alcohol, cetyl alcohol, cetostearyl alcohol, stearyl alcohol, octamethylcyclotetrasiloxane, mineral oil, spindle oil, tamanu oil, or any combination thereof.

7. The method of claim 1, wherein the cellulosic substrate comprises hardwood kraft fibers, softwood kraft fibers, hardwood sulfite fibers, softwood sulfite fibers, or any combination thereof.

8. The method of claim 1, the gel softener further comprising a surfactant in a range between about 10 wt. % and about 98 wt. % based on the total weight of the gel softener.

9. The method of claim 8, wherein the surfactant is hydroxyethylglycinate coco-glucosides crosspolymer, hydroxyethylglycinate laurylglucosides crosspolymer, or any combination thereof.

10. A method of making a tissue comprising:
forming a cellulosic substrate; and
depositing a gel softener onto the substrate, the softener being sprayable at a temperature above room temperature and forming into a sold or semi-solid state at room temperature, the gel softener comprising a contact product of a gelator and a pharmaceutically or cosmetically acceptable oil, the gelator being a mannitol octyl diester, a sorbitol octyl diester, or 1,3:2,4 dibenzylidene sorbitol,
the mannitol octyl diester having the following structure: 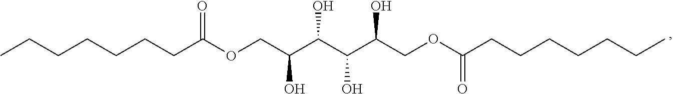 [SMILES: CCCCCCCC(=O)OC[C@H](O)[C@H](O)[C@@H](O)[C@@H](O)COC(=O)CCCCCCC]
and the sorbitol octyl diester having the following structure: 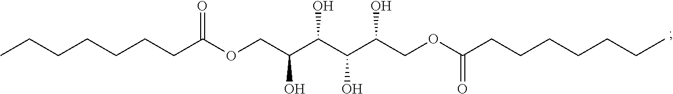 [SMILES: CCCCCCCC(=O)OC[C@@H](O)[C@H](O)[C@@H](O)[C@@H](O)COC(=O)CCCCCCC]
and the gel softener being formed on the substrate by either:
(i) spraying the oil onto the substrate to form an oil layer, and then spraying the mannitol octyl diester or the sorbitol octyl diester onto the oil layer to form the gel softener, or
(ii) mixing the 1,3:2,4 dibenzylidene sorbitol with the oil at a temperature greater than room temperature to form the gel softener, and then spraying the gel softener onto the substrate.

11. The method of claim 10, wherein the temperature of the gel softener is greater than about 105° C.

12. The method of claim 10, wherein the gelator is present in the gel softener as a ratio of % weight to volume of oil (w/v) in a range between about 1.5% (w/v) and about 5.0% (w/v).

13. The method of claim 10, wherein the gel softener is deposited onto the cellulosic substrate at an add-on level in a range between about 1 pound per ton of tissue and about 60 pounds per ton of tissue.

14. The method of claim 10, wherein the oil is an emollient, a humectant, a glycerol ester of fatty acids, a fatty acid alcohol, an essential oil, a C₁₂-C₁₅ alkyl benzoate ester, or any combination thereof.

15. The method of claim 10, wherein the oil is castor oil, olive oil, corn oil, carnation oil, soy bean oil, tubaki oil, cottonseed oil, sesame oil, avocado oil, jojoba oil, safflower oil, apricot oil, evening primrose oil, rose hip oil, grapeseed oil, coconut oil, carrot seed oil, eucalyptus oil, chamomile oil, neroli oil, tea tree oil, ylang ylang oil, spearmint oil, lavender oil, peppermint oil, sandalwood oil, squalane, mink oil, turtle oil, emu oil, cod liver oil, orange roughy oil, mink oil, polybutene, isopropyl myristate, isocetyl myristate, cetylisooctansate, isostearic acid, lauric acid, oleic acid, polyethylene glycol, polyethylene glycol 400, polyethylene glycol 860 monooleate, propylene glycol, glycerol, methylene glycol, polypropylene glycol, Guerbet ester, isostearyl alcohol, oleyl alcohol, cetyl alcohol, cetostearyl alcohol, stearyl alcohol, octamethylcyclotetrasiloxane, mineral oil, spindle oil, tamanu oil, or any combination thereof.

16. The method of claim 10, wherein the cellulosic substrate comprises hardwood kraft fibers, softwood kraft fibers, hardwood sulfite fibers, softwood sulfite fibers, or any combination thereof.

17. The method of claim 10, the gel softener further comprising a surfactant in a range between about 10 wt. % and about 98 wt. % based on the total weight of the gel softener.

18. The method of claim 17, wherein the surfactant is hydroxyethylglycinate coco-glucosides crosspolymer, hydroxyethylglycinate laurylglucosides crosspolymer, or any combination thereof.

19. A method of making a tissue comprising:
forming a cellulosic substrate; and
depositing a gel softener onto the substrate, the gel softener being sprayable at a temperature above room temperature and forming into a solid or semi-solid state at room temperature, the gel softener comprising a contact product of a gelator and a pharmaceutically or cosmetically acceptable oil, the gelator being a sugar diester or 1,3:2,4 dibenzylidene sorbitol, the sugar diester having the following structure: 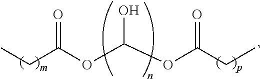
wherein n is an integer from 4 to 24, m is an integer from 6 to 32, p is an integer from 6 to 32, and m and p are the same or different; and
the gel softener being formed on the substrate by either:
(i) spraying the oil onto the substrate to form an oil layer, and then spraying the sugar diester onto the oil layer to form the gel softener, or
(ii) mixing the 1,3:2,4 dibenzylidene sorbitol with the oil at a temperature above room temperature to form the gel softener, and then spraying the gel softener onto the substrate.

20. The method of claim 19, wherein the temperature of the gel softener is greater than about 105° C.

21. The method of claim 19, wherein the gelator is present in the gel softener as a ratio of % weight to volume of oil (w/v) in a range between about 1.5% (w/v) to about 5.0% (w/v).

22. The method of claim 19, wherein the gel softener is deposited onto the cellulosic substrate at an add-on level in a range between about 1 pound per ton of tissue and about 60 pounds per ton of tissue.

23. The method of claim 19, wherein the oil is an emollient, a humectant, a glycerol ester of fatty acids, a fatty acid alcohol, an essential oil, a C₁₂-C₁₅ alkyl benzoate ester, or any combination thereof.

24. The method of claim 19, wherein the oil is castor oil, olive oil, corn oil, carnation oil, soy bean oil, tubaki oil, cottonseed oil, sesame oil, avocado oil, jojoba oil, safflower oil, apricot oil, evening primrose oil, rose hip oil, grapeseed oil, coconut oil, carrot seed oil, eucalyptus oil, chamomile oil, neroli oil, tea tree oil, ylang ylang oil, spearmint oil, lavender oil, peppermint oil, sandalwood oil, squalane, mink oil, turtle oil, emu oil, cod liver oil, orange roughy oil, mink oil, polybutene, isopropyl myristate, isocetyl myristate, cetylisooctansate, isostearic acid, lauric acid, oleic acid, polyethylene glycol, polyethylene glycol 400, polyethylene glycol 860 monooleate, propylene glycol, glycerol, methylene glycol, polypropylene glycol, Guerbet ester, isostearyl alcohol, oleyl alcohol, cetyl alcohol, cetostearyl alcohol, stearyl alcohol, octamethylcyclotetrasiloxane, mineral oil, spindle oil, tamanu oil, or any combination thereof.

25. The method of claim 19, wherein the cellulosic substrate comprises hardwood kraft fibers, softwood kraft fibers, hardwood sulfite fibers, softwood sulfite fibers, or any combination thereof.

26. The method of claim 19, the gel softener further comprising a surfactant in a range between about 10 wt. % and about 98 wt. % based on the total weight of the gel softener.

27. The method of claim 26, wherein the surfactant is hydroxyethylglycinate coco-glucosides crosspolymer, hydroxyethylglycinate laurylglucosides crosspolymer, or any combination thereof.
